SEQUENCE LISTING (12 sequences)

SEQ ID NO: 1 - protein, 469 aa, Solanum tuberosum
MAPEFQKNAMYVSNELENGDVQKNFDDDGREKRTGTLLTASAHIITAVIGSGVLSLAWAIAQLGWVAGPAVLFAFSFITYFTSTLLADCYRSPGPISGKRNYTYMDVVRSHLGGVKVTLCGIAQYANLVGVTIGYTITASISMVAVKRSNCFHKNGHEASCSIESYPYMIIFAVIQIVLSQIPNFHKLSWLSILAAVMSFTYASIGLGLSIAKASGVGHHVKTALTGVVVGVDVSGTEKVWRSFQAIGDIAFAYAYSTVLIEIQDTLKSSPSESKVMKRASLAGVSTTTLFYVLCGTIGYAAFGNNAPGNFLTGFGFYEPFWLIDFANVCIAVHLVGAYQVFCQPLYGFVEGRCSERWPDSKFITSEYAMQVPWCGTYNLNLFRLVWRTTYVIVTAVIAMIFPFFNDFLGLIGAASFYPLTVYFPIEMYIAQRKIPKYSFTWVWLKILSWTCLISITCCSCWIDPGTCH

SEQ ID NO: 2 - DNA, 1410 bp, Solanum tuberosum
  source 1..1410: mol_type = genomic DNA
atggcacctgaatttcaaaagaacgctatgtacgtctcaaatgaactcgaaaatggagacgttcaaaaaaactttgatgatgatggacgtgaaaaaagaacagggactttgttgactgctagtgcacatattattactgctgtgattggttcaggagtactttcactagcatgggctatagctcaattaggatgggtggctggtcctgctgttctctttgccttttctttcatcacatatttcacttccacacttcttgccgactgttacaggtctccgggaccgatttccggcaagagaaactatacttatatggacgttgtccgctctcacttaggaggtgtgaaggtgacattgtgtggaattgcacaatatgctaaccttgttggagttacaattggatatacaattacagcatctattagtatggtggctgtaaagaggtcaaattgttttcacaaaaatgggcatgaagcaagttgctcaattgaaagttacccatacatgataatatttgcagtaattcaaatagttcttagccaaattccaaatttccacaagctctcatggttatcaattcttgctgctgttatgtcatttacttatgcttctattggtcttggactatccatagccaaagcctcaggtgtggggcaccatgtgaagacagccctaacaggggtggtagtaggtgtggatgtatctggaacagaaaaagtatggagaagtttccaagctattggagatattgcatttgcttatgcttattccacagttcttattgaaatacaggacacactgaaatcatcaccatcagaaagcaaggtaatgaagagagcctcactagctggagtttccaccacaactttattctatgtactttgtggtaccattggctatgcagcctttggaaacaatgctccaggaaattttcttactggatttggtttctatgaacctttttggctaattgactttgccaacgtctgcatcgctgtccaccttgtcggagcttatcaggttttctgccaaccattatatggattcgtggagggtcgttgcagcgaacgatggccagacagcaaattcataacctcagaatacgcgatgcaagttccatggtgtggcacttacaacctcaacttattcaggctggtgtggagaacaacatatgttatagtgacagcggtgattgcaatgatattccccttcttcaacgacttcttggggttgatcggagcagcatcgttctatccattaaccgtttacttcccaatagagatgtacattgctcagagaaagataccaaagtattctttcacatgggtatggctgaaaatattgagttggacttgtttgatcagtatcacttgttgcagctgctggatcgatccagggacttgccactga

SEQ ID NO: 3 - protein, 449 aa, Solanum tuberosum
MGTQAPSNYDDSKIDTRTEEEKAIDAWLPITSSRNAKWWYSAFHNVTAMVGAGVLSLPYAMSELGWGPGVTVMVVSWIITLYTLWQMVEMHEMIPGKRFDRYHELGQHAFGEKLGLWIVVPQQLIVEVGVDIVYMVTGGKSLQKVHQLVCKPEDGCADIKLSYFIMIFASVHFVLSHLPNFNSISGVSLAAAVMSLSYSTIAWGASVKKGVQPNVDYGYKAHSTAGTVFDFLSGLGEVAFAYAGHNVVLEIQATIPSTPDKPSKVPMWRGVVVAYIVVALCYFPVAFIGYWMFGNAVEDNILMSLNKPTWLIIMANMFVVVHVIGSYQIYAMPVFDMIETVLVKKLRFKPTWYLRFVTRNIYVAFTMFVGITFPFFGGLLGFFGGFAFAPTTYFLPCIMWLAIYKPRRWSLSWIANWICIIFGVLLMVLAPIGGLRSIIVQAKTYKFYN

SEQ ID NO: 4 - DNA, 1350 bp, Solanum tuberosum
  source 1..1350: mol_type = genomic DNA
atgggaactcaagctccatcaaattacgatgactccaagattgatacaagaacagaagaagagaaggcaattgatgcgtggcttccgattacttcttcgaggaatgcaaaatggtggtattcagcttttcacaatgttactgctatggttggtgctggtgtcctcagtcttccttatgccatgtctgagcttggatggggacctggtgtaactgtgatggttgtatcttggattatcactttatacacactatggcaaatggttgaaatgcacgaaatgattccagggaaacgttttgatagatatcatgagttggggcaacatgcttttggggaaaagcttggattatggattgttgttccccaacaactaattgtcgaagttggtgttgacattgtttatatggtcaccggaggaaaatcacttcagaaggtgcaccaattggtttgcaaaccagaagacggctgcgcagatatcaaactttcatacttcatcatgatatttgcctctgtccattttgtgctatctcatcttcccaatttcaattccatatctggtgtgtctttggcagcagctgttatgtccttaagttactctacaattgcttggggagcatcagttaagaagggtgtacaaccaaatgtggattacgggtacaaggcacacagcactgcaggaactgtgtttgattttctaagcggattgggagaagtggcttttgcatacgcgggtcataatgtggtgttggagattcaagctacaattccttcaacacctgacaaaccttccaaagtacctatgtggaggggagttgttgttgcttacatagttgtggccctctgttacttccctgttgcttttattggatactggatgtttggaaatgcagtggaagacaacattctcatgtctttgaacaaacctacctggctcattatcatggctaacatgtttgtcgttgtccatgttattggaagctatcagatctatgcaatgccagtgtttgacatgattgagaccgtgcttgttaagaaacttaggttcaagcctacttggtatttgcgatttgttaccagaaacatctatgttgctttcacaatgtttgttggaatcaccttccctttctttggtggccttcttggattctttggaggatttgcttttgccccaacaacatattttctgccttgcatcatgtggctagcaatctacaagccaaggagatggagtctctcttggattgctaattggatttgcattatttttggagttttgttgatggttttagcaccaattggtgggttgagatccatcatagtacaagcaaagacctacaaattttacaattag

SEQ ID NO: 5 - protein, 473 aa, Solanum tuberosum
MTMADKAHQVFEVYGESKCFDDDGRIKRTGSVWTASAHIITAVIGSGVLSLAWATAQLGWVAGPTVLLLFSFVTYYTSALLSDCYRTGDPVTGKRNYTYMDAVRANLGGFQVKICGVIQYANLFGVAIGYTIAASISMVAVNRSNCFHKQGHRAACNVSSTPYMIIFGVMEIIFSQIPDFDQISWLSIVAAVMSFTYSTIGLGLGVAQVAETGKIEGSLTGISIGTEVTEMQKIWRSFQALGAIAFAYSYSLILIEIQDTLKSPPAEAKTMKRATLISVAVTTVFYMLCGCFGYAAFGDQSPGNLLTGFGFYNPYWLLDIANVAIVVHLVGAYQVYCQPLFAFVEKTATEWYPDSKIITKEIDVPIPGFKPFKLNLFRLVWRTIFVIITTVISMLMPFFNDVVGILGAFGFWPLTVYFPVEMYIVQKRITKWSGRWICLQILSGACLVISIAAAAGSFAGVASDLKVYRPFQS

SEQ ID NO: 6 - DNA, 1361 bp, Solanum tuberosum Tst1 virus
  source 1..1361: mol_type = genomic DNA
atgacgatggtcgaatcaaaaggactgggtctgtttggacggcaagtgctcacatcataactgctgtgataggatcaggagttttatcattggcttgggctactgctcagcttggatgggttgctggtccaaccgtattgcttctattctcctttgttacttactatacctctgctttgctttccgattgttaccggaccggtgatccagttaccggaaaaagaaattatacttatatggatgctgttcgagccaatcttggtggatttcaagttaagatttgtggtgtaattcaatatgcaaatctttttggagttgcaattggttataccatcgcggcttccattagcatggtggctgtgaatagatctaattgtttccataaacaaggtcatcgtgctgcttgcaatgtttcaagcactccctacatgatcatttttggagtaatggaaattatcttctcacaaatcccagatttcgatcagatttcttggctttctattgttgctgctgttatgtcctttacttactctacaattggtcttggtttaggagttgctcaagttgcagaaacaggaaaaattgaaggaagtttaactgggattagcattggaactgaagtaactgaaatgcagaagatttggagaagctttcaagctcttggagctatagcttttgcttattcttactccctaatccttatcgaaattcaggatacactcaaatcaccaccagcagaagcaaagacaatgaaaagggcaacactaattagtgtggcagtaacaacagttttctacatgctttgtggttgctttgggtatgcagcatttggagatcaatcccctggaaacctactaaccggattcggattctacaacccctattggcttcttgacatcgcaaatgtagccatcgttgttcacctagtaggtgcctaccaagtttactgccaacccctttttgcctttgttgaaaaaacagcaactgaatggtaccctgacagcaaaatcattacaaaagagatcgatgtaccaatcccgggatttaagcctttcaagctcaaccttttccgtctagtctggaggacaatattcgtgatcatcaccacagttatatcaatgttaatgccattctttaacgacgtcgttggcattcttggagcttttggattttggccacttacagtatacttcccagtagaaatgtacattgtgcaaaagagaattaccaaatggagtggaagatggatatgtcttcaaatacttagtggtgcttgccttgttatctcaattgctgcagctgctggttcttttgctggagttgcttctgatttaaaagtttacaggccttttcagagttaa

SEQ ID NO: 7 - protein, 487 aa, Triticum aestivum
MEKKQASYGKGAARMAPMEVSVEAGNGRESDWLDDDGRPRRSGTVWTASAHIITAVIGSGVLSLAWAIAQLGWVAGPAIMLLFALVIYYTSTLLAECYRSGDPETGKRNYTYMDAVRSYLPGTKVKLCGVIQYANLVGVAIGYTIAASISMRAIGRADCFHYHDVRGRSGKDECKSSSNPYMIVFGVVQILFSQIPDFDQIWWLSIVAAVMSFTYSTIGLGLGIAQTVANGGIQGSLTGLSVGPGVTSMQKVWRSLQAFGNIAFAYSYSIILIEIQDTVKAPPPSEAKVMKKATGISVATTTVFYMLCGCMGYAAFGDAAPDNLLTGFGFYEPFWLLDVANAAIVLHLVGAYQVFCQPLFAFVEKWAAARWPDSAFIARELRVGPFAISVFRLTWRTAFVCLTTVVSMLLPFFGDVVGLLGAVAFWPLTVYFPVEMYIVQRGVRRGSTRWVCLKMLSAACLVVSVAAAAGSIADVIGELKEYRPFSG

SEQ ID NO: 8 - DNA, 1464 bp, Triticum aestivum
  source 1..1464: mol_type = genomic DNA
atggagaagaagcaggcgagctacgggaagggcgcggccaggatggcgccgatggaggtgtcggtggaggccgggaacggcagggagtccgactggcttgacgacgacgggcgcccgcgtcggtcgggcacggtgtggacggcgagcgcccacatcatcaccgccgtcatcggctcgggcgtcctctcgctggcgtgggccatcgcccagctcggctgggtggcgggccccgccatcatgctcctgttcgcgctcgtcatctactacacctccacgctcctcgccgagtgctaccgctccggcgacccggagaccggcaagcgcaactacacctacatggacgccgtccgctcctacctcccgggcaccaaggtgaagctctgcggcgtcatccagtacgccaacctcgtcggcgtcgccatcggctacaccatcgccgcctccatcagcatgcgggcgatcgggagggcggactgcttccattaccacgacgtcaggggccgcagcgggaaggacgagtgcaagagctccagcaacccgtacatgatcgtcttcggggtcgtgcagatcctcttctcgcagataccggacttcgaccagatatggtggctctccatcgtcgccgccgtcatgtccttcacctactccaccatcgggctcggcctcggcatcgcgcagaccgtcgccaacggtggcatccagggcagcctcaccggcctcagcgtcggcccgggcgtgacctccatgcagaaggtgtggcgcagccttcaggccttcggcaacattgccttcgcctactcctactccatcatcctcatcgaaatccaggacacggtgaaggcgccgccgccgtcggaggcgaaggtgatgaagaaggctacggggataagcgtggcgacgacgacggtgttctacatgctgtgcgggtgcatggggtacgcggcgttcggcgacgctgcgcccgacaacctcctcacggggttcggcttctacgagcccttctggctgctggacgtcgccaacgccgccatcgtgctgcacctcgtcggcgcctaccaggtcttctgccagcccctcttcgccttcgtcgagaagtgggcggcggcgaggtggcccgacagcgccttcatcgcccgggagctccgcgtgggacccttcgccatcagcgtcttccggctcacctggcgcacggccttcgtctgcctcaccaccgtcgtctccatgctgctccccttcttcggcgacgtcgtggggctcctcggcgccgtcgcgttctggccgctcaccgtctacttccccgtcgagatgtacatcgtgcagcgcggcgtgcgacgggggagcacgcggtgggtttgcctcaagatgctcagcgccgcctgcctcgtcgtgtccgtcgccgccgcggccgggtccatagccgacgtcatcggcgagctcaaggagtaccggccgttcagcggctga

SEQ ID NO: 9 - protein, 487 aa, Triticum dicoccoides; identical to SEQ ID NO: 7

SEQ ID NO: 10 - DNA, 1464 bp, Triticum dicoccoides
  source 1..1464: mol_type = genomic DNA
atggagaagaagcaggcgagctacgggaagggcgcggccaggatggcgccgatggaggtgtcggtggaggccgggaacggcagggagtccgactggcttgacgacgacgggcgcccgcgtcggtcgggcacggtgtggacggcgagcgcccacatcatcaccgccgtcatcggctcgggcgtcctctcgctggcgtgggccatcgcccagctcggctgggtggcgggccccgccatcatgctcctgttcgcgctcgtcatctactacacctccacgctcctcgccgagtgctaccgctccggcgacccggagaccggcaagcgcaactacacctacatggacgccgtccgctcctacctcccgggcaccaaggtgaagctctgcggcgtcatccagtacgccaacctcgtcggcgtcgccatcggctacaccatcgccgcctccatcagcatgcgggcgatcgggagggcggactgcttccattaccacgacgtcaggggccgcagcgggaaggacgagtgcaagagctccagcaacccgtacatgatcgtcttcggggtcgtgcagatcctcttctcgcagataccggacttcgaccagatatggtggctctccatcgtcgccgccgtcatgtccttcacctactccaccatcgggctcggcctcggcatcgcgcagaccgtcgccaacggtggcatccagggcagcctcaccggcctcagcgtcggcccgggcgtgacctccatgcaaaaggtgtggcgcagccttcaggccttcggcaacattgccttcgcctactcctactccatcatcctcatcgaaatccaggacacggtgaaggcgccgccgccgtcggaggcgaaggtgatgaagaaggcgacggggataagcgtggcgacgacgacggtgttctacatgctgtgcgggtgcatggggtacgcggcgttcggcgacgcggcgcccgacaacctcctcacggggttcggcttctacgagcccttctggctgctggacgtcgccaacgccgccatcgtgctgcacctcgtcggcgcctaccaggtcttctgccagcccctcttcgccttcgtcgagaagtgggcggcggcgaggtggcccgacagcgccttcatcgcccgggagctccgcgtgggacccttcgccatcagcgtcttccggctcacctggcgcacggccttcgtctgcctcaccaccgtcgtctccatgctgctccccttcttcggcgacgtcgtggggctcctcggcgccgtcgcgttctggccgctcaccgtctacttccccgtcgagatgtacatcgtgcagcgcggcgtgcgacgggggagcacgcggtgggtttgcctcaagatgctcagcgccgcctgcctcgtcgtgtccgtcgccgccgcggccgggtccatagccgacgtcatcggcgagctcaaggagtaccggccgttcagcggctga

SEQ ID NO: 11 - protein, 482 aa, Pisum sativum
MVVEKNASKNHHHQTFDVSIDQQLDSKFFDDDGRVKRTGTSWTASAHVITAVIGSGVLSLAWAIAQLGWIAGPVVMILFAWVTYYTSVLLAECYRNGDPVNGKRNYTYMEVVHSNLGGLQVQFCGFIQYLNLIGVAIGYTVASAISMMAIERSNCYHRSGGKDPCHMNSNAYMIAFGAVQIIVSQIPDFDQLWWLSIVAAVMSFTYSTIGLGLGIGKVMENKKFAGTITGVNDVTKAQKTWGSLQALGDIAFAYSFSMILIEIQDTVKAPPPSESKTMKKATLISVIVTTFFYMLCGCLGYAAFGNSSPGNLLTGFGFYNPFWLLDIANAAIVIHLIGAYQVYCQPLYAFVENYMVKRFPDNYFLNKNIKIPIPGLDMYKLNLFKLVWRTVFVILTTLVSMLLPFFNDIVGLLGALGFWPLTVYFPVEMYIIQKKIPKWSTKWTCLQLLSGACLIITIAASVGSIAGIYLDLKVFKPFKTIY

SEQ ID NO: 12 - DNA, 1449 bp, Pisum sativum
  source 1..1449: mol_type = genomic DNA
atggttgttgagaagaatgctagcaagaatcatcatcaccaaacatttgatgtatccattgatcaacaactcgattctaagtttttcgatgatgacggtcgcgtcaaacgaaccggcacatcttggactgcaagtgcccatgtaataacagcagtaattggctctggagtgctgtctctagcttgggctatagctcagctgggatggattgctggtcctgttgtcatgatcctctttgcttgggttacttattacacctcggttcttctcgctgaatgttatcgtaatggtgatccagtaaatggcaagagaaactacacttacatggaagttgttcattcgaatcttggtggtttacaagtccagttttgtgggttcattcagtatctcaaccttattggggtcgcgattggatacacggtagcttctgccataagcatgatggcgattgaaaggtcaaattgttatcataggagtggaggaaaagatccatgtcatatgaacagcaacgcttacatgattgcttttggtgctgtgcaaattatagtctctcaaattccagattttgatcagttatggtggctttccattgttgctgctgttatgtcctttacatattcgacgattggtctaggccttggtatcggaaaagttatggagaacaaaaagtttgctggaaccattactggagtaaatgatgtgacaaaagctcagaaaacttgggggagtttgcaagctcttggtgacatagcttttgcttattcattctccatgattcttatagaaattcaggacacggtaaaagcaccaccaccgtcagagtcgaaaaccatgaagaaggctacattgatcagtgttatagtgacaacatttttctacatgctttgcggttgtttaggatacgcagcgtttggaaattcaagtcccggaaaccttttaaccggtttcggattctataacccattttggctccttgacatagccaatgctgccatagtgattcaccttattggtgcataccaagtctattgccaacccctttatgctttcgtcgaaaactacatggtgaaaagatttccagacaattattttttgaacaagaacataaagattccaatcccgggtttagatatgtataaactcaaccttttcaaattggtttggaggacagtttttgtaatattaacaactttggtctcaatgcttcttccattcttcaatgatattgttggacttcttggtgcacttggattttggcctctaacggtttattttccggtggagatgtatataattcaaaagaaaataccaaagtggagcacaaaatggacatgccttcaattgcttagtggtgcttgtcttataatcactatagcagcttcagttggttctattgctgggatttatcttgatcttaaagtgtttaagccattcaagactatctattga

(19) United States
(12) Patent Application Publication
(10) Pub. No.: US 2026/0055421 A1
(43) Pub. Date: Feb. 26, 2026

(54) COMPOSITIONS AND METHODS FOR INCREASING THE AMINO ACID AND PROTEIN CONTENT IN STORAGE ORGANS OF PLANTS

(71) Applicant: POLOPO LTD., D.N. Emek Sorek (IL)

(72) Inventors: Raya LIBERMAN-ALONI, Tel-Aviv (IL); Maya SAPIR-MIR, D.N. Emek Sorek (IL)

(21) Appl. No.: 18/995,073

(22) PCT Filed: Jul. 30, 2023
(86) PCT No.: PCT/IL2023/050784

§ 371 Date: Jan. 15, 2025

Related U.S. Application Data
(60) Provisional application No. 63/393,923, filed on Jul. 31, 2022.

(51) Int. Cl. C12N 15/82; C07K 14/415

(52) U.S. Cl. CPC C12N 15/8226; C07K 14/415; C12N 15/8251

(57) ABSTRACT

The present invention provides methods and compositions for increasing amino acids content and proteins in plant storage organs. In particular, the present invention relates to transgenic Solarium tuberosum plants overexpressing amino acid permease (AAP) and/or lysin histidine transporter (LHT), the plants store high amount of essential amino acids and capable of producing high amount of heterologous proteins in the tubers.

### FIELD OF THE INVENTION

The present invention relates to methods and compositions for increasing amino acid content and protein amount in plant storage organs. In particular, the present invention relates to transgenic tuberous plants overexpressing at least one amino acid permease (AAP) and/or at least one lysin histidine transporter (LHT), the plants storing high amount of essential amino acids and capable of producing high amounts of heterologous proteins in the tubers.

### BACKGROUND OF THE INVENTION

Producing valuable proteins including pharmaceutical, nutritional, cosmetical and industrial proteins in plants offers advantages over some other manufacturing methods, notably the low cost and case of scaling up production to meet demand. Unlike microbial fermentation, plants are capable of carrying out post-translational modifications and, unlike production systems based on mammalian cell cultures, plants are devoid of human infective viruses and prions. A large panel of strategies and new plant expression systems were developed to improve the plant-made recombinant protein's yields and quality.

The tubers of the potato plant (Solanum tuberosum) are a common, primary food worldwide. In addition to carbohydrates, potato tubers provide a source of protein in the diet. The amount of total proteins is low, however they are rich in lysine and sulfur-containing amino acids. The proteinase inhibitor I and proteinase inhibitor II are abundant, water-soluble, storage proteins that are present in potato tubers, where their levels have been shown to closely correlate with the amount of total protein in the tubers. The proteins denature readily when cooked, and become excellent food proteins.

In plants, leaves are the predominant sites for amino acid biosynthesis. Amino acids are exported from leaves through the phloem to supply other organs, such as roots and tubers. Concentration and composition of amino acids in phloem and mesophyll cytosol are similar, whereas a concentration gradient can be found between the apoplast and phloem sap. This gradient indicates that active transport systems are involved in uptake of amino acids from the apoplast into the phloem. The similar concentration and composition of amino acids in the phloem and the mesophyll cells points to non-selective uptake systems. Physiological studies with leaf membrane vesicles support the hypothesis of lowly selective uptake systems for amino acids involving a proton-coupled mechanism.

Amino acid transporter encoding genes have been isolated from Arabidopsis by complementation of yeast transport mutants with plant cDNAs. These genes can be classified into different families. The ATF-super family (amino acid transporter family) includes several subfamilies such as the amino acid permeases (AAPs), the lysin histidine transporters (LHTs), the proline and compatible solutes transporters (ProTs), and the putative auxin transporter (AUXs). One transporter, ANT1, has been characterized as a transporter for neutral and aromatic amino acids. The cationic amino acid transporters (CATs) are members of the second plant amino acid transporter family, namely the APC-superfamily. Eight members of the AAP family (AAP1-8) were shown to mediate transport of a broad spectrum of amino acids with low selectivity by a proton-coupled mechanism. For glutamine, glutamate and asparagine as major transport forms of organic nitrogen, these transporters have reasonable Kₘ values with respect to the concentration of these amino acids in the phloem and xylem. Aspartate is transported by most AAPs with an extremely low affinity and efficiency. Properties of AAPs thus match the activities expected for protein transport involved in moving a wide spectrum of amino acids into the phloem sap, and are consistent with the properties described for uptake kinetics measured in plasma membrane vesicles of leaves. Taken together, the transporters of the AAP family fulfill the criteria of low selectivity together with a proton-coupled mechanism predicted to play a role in phloem loading (Koch et al. The Plant Journal (2003) 33, 211-220).

US Patent Application Publication No. 20050223441 discloses potato tubers that each include water-soluble protein at a concentration of at least 5 mg/ml. The application discloses potato tubers that include an exogenous nucleic acid molecule encoding a systemin molecule, or a prosystemin molecule, that is expressed in the potato tubers.

US Patent Application Publication No. 20070130643 discloses transgenic plants which accumulate free amino acids, particularly at least one amino acid selected from among glutamic acid, asparagine, aspartic acid, serine, threonine, alanine and histidine accumulated in a large amount, in the edible parts of the plants. US20070130643 further discloses a method of increasing the yielding of potato and to provide a transgenic potato of which yielding can be increased. The method comprises introducing a sequence encoding glutamate dehydrogenase (GDH) gene into a plant together with a suitable regulatory sequence to express it in a plant cell, and thereby the GDH gene is excessively expressed.

There is still an unmet need for improved and robust methods for enhancing the amount of amino acids and proteins in plant's storage organs, such as tubers.

### SUMMARY OF THE INVENTION

The present invention provides methods and compositions for increasing the amount of free essential amino acids and of proteins in plant storage organs such as tubers, seeds, fruit and roots. The methods of the invention in some embodiments enable the enrichment of essential amino acids in tubers. In additional embodiments, the enrichment of essential free amino acids enables the high production of recombinant proteins in tubers.

It is now disclosed that overexpression of AAP and/or LHT is capable of increasing the nutritional value of plant's storage organs, in particular tubers. The transgenic plants described herein in some embodiments accumulate essential amino acids as well as total endogenous proteins in an increased amount compared to corresponding non-transgenic plants. Furthermore, the enrichment of the amino acids in the storage organs of plants overexpressing AAP or LHT, increases the amount of expressed heterologous proteins in said organs.

According to one aspect, the present invention provides a plant having an edible storage organ, the plant comprises at least one cell having enhanced expression and/or activity of at least one amino acid permease (AAP) and/or at least one Lysin histidine transporter (LHT), compared to the expression and/or activity in a corresponding control plant and having: (1) an increased amount of at least one free amino acid in the plant storage organ, and/or (2) an increased amount of at least one protein in said organ; compared to the amount in the corresponding control plant.

According to some embodiments, the storage organ is selected from the group consisting of a tuber, root, seed, corm, rhizome, and fruit. Each possibility represents a separate embodiment of the invention. According to certain embodiments, the storage organ is a tuber.

According to some embodiments, the plant is a tuberous plant. According to certain embodiments, the plant is selected from the group consisting of Solanum tuberosum, S. demissum, S. acaule, S. stoloniferum, S. phureja, S. gonicalyx, S. stenotomum, S. berthaultii, S. brevicaule, S. bukasovii, S. canasense, S. gourlayi, S. leptophyes, S. multidissectum, S. oplocense, S. sparsipilum, S. sucrense, S. venturii, and S. vernei. Each possibility represents a separate embodiment of the invention. According to certain exemplary embodiments, the plant is Solanum tuberosum (potato).

According to other embodiments, the plant is Cassava.

According to certain embodiment, the plant is genetically engineered to have enhanced expression and/or activity of the at least one amino acid permease (AAP) and/or the at least one Lysin histidine transporter (LHT).

According to some embodiments, the plant is a transgenic plant.

According to some embodiments, the plant comprises at least one cell having an enhanced expression and/or activity of LHT. According to some embodiments, the plant comprises at least one cell having an enhanced expression and/or activity of AAP.

According to some embodiments, the AAP is selected from the group consisting of amino acid permease 1 (AAP1), amino acid permease 3 (AAP3), Triticum aestivum amino acid permease 13 (TaAAP13), Triticum dicoccoides amino acid permease 3 (TdAAP3), Pisum sativum amino acid transporter (PsAAP1), Oryza sativa AAP (OsAAP16) and Arabidopsis thaliana AAP1 (AtAAP1). According to some embodiments, the plant comprises at least one cell having an enhanced expression and/or activity of AAP1. According to some embodiments, the plant comprises at least one cell having an enhanced expression and/or activity of AAP3. According to some embodiments, the plant comprises at least one cell having an enhanced expression and/or activity of TaAAP13. According to some embodiments, the plant comprises at least one cell having an enhanced expression and/or activity of TdAAP3. According to some embodiments, the plant comprises at least one cell having an enhanced expression and/or activity of PsAAP1.

According to certain embodiments, the LHT is selected from the group consisting of LHT1 and LHT2. Each possibility represents a separate embodiment of the present invention.

According to additional embodiments, the plant comprises at least one cell having an enhanced expression and/or activity of both AAP and LHT.

According to some embodiments, the plant comprises at least one cell having an enhanced expression and/or activity of at least one of AAP1, AAP3, TaAAP13, TdAAP3, PsAAP1 and of LHT1 and LHT2. According to certain embodiments, the plant comprises at least one cell having an enhanced expression and/or activity of at least one of StAAP1 and StAAP3. According to certain embodiments, the plant comprises at least one cell having an enhanced expression and/or activity of at least one of LHT1 and LHT2.

According to some embodiments, the AAP protein comprises an amino acid sequence having at least 75%, or at least 80%, or at least 85%, or at least 90%, or at least 95%, at least 98%, or 100% identity to the amino acid sequence of Solanum tuberosum AAP1. According to certain embodiments, the Solanum tuberosum AAP1 comprises the amino acid sequence set forth in SEQ ID NO: 1.

According to some embodiments, the polynucleotide encoding the AAP protein comprises a nucleotide sequence having at least 60%, 65%, 70%, 75%, 80%, 85%, 90%, 95%, 98%, or 100% identity to the nucleotide sequence of the open reading frame of Solanum tuberosum aap1. According to some embodiments, the open reading frame of Solanum tuberosum aap1 comprises the nucleotide sequence of SEQ ID NO: 2.

According to some embodiments, the LHT1 protein comprises an amino acid sequence having at least 75%, or at least 80%, or at least 85%, or at least 90%, or at least 95%, or at least 98%, or 100% identity to the amino acid sequence of Solanum tuberosum LHT1. According to certain embodiments, the Solanum tuberosum LHT1 comprises the amino acid sequence of SEQ ID NO: 3.

According to some embodiments, the polynucleotide encoding the LHT1 protein comprises a nucleotide sequence having at least 60%, 65%, 70%, 75%, 80%, 85%, 90%, 95%, 98%, or 100% identity to the nucleotide sequence of the open reading frame of Solanum tuberosum lht1. According to some embodiments, the open reading frame of Solanum tuberosum lht1 comprises the nucleotide sequence of SEQ ID NO: 4.

According to some embodiments, the AAP protein comprises an amino acid sequence having at least 75%, or at least 80%, or at least 85%, or at least 90%, or at least 95%, at least 98%, or 100% identity to the amino acid sequence of Solanum tuberosum AAP3. According to certain embodiments, the Solanum tuberosum AAP3 comprises the amino acid sequence set forth in SEQ ID NO: 5.

According to some embodiments, the polynucleotide encoding the AAP protein comprises a nucleotide sequence having at least 60%, 65%, 70%, 75%, 80%, 85%, 90%, 95%, 98%, or 100% identity to the nucleotide sequence of the open reading frame of Solanum tuberosum aap3. According to some embodiments, the open reading frame of Solanum tuberosum aap3 comprises the nucleotide sequence of SEQ ID NO: 6.

According to some embodiments, the AAP protein comprises an amino acid sequence having at least 75%, or at least 80%, or at least 85%, or at least 90%, or at least 95%, at least 98%, or 100% identity to the amino acid sequence of Triticum aestivum AAP13. According to certain embodiments, the Triticum aestivum AAP13 comprises the amino acid sequence set forth in SEQ ID NO: 7.

According to some embodiments, the polynucleotide encoding the AAP protein comprises a nucleotide sequence having at least 60%, 65%, 70%, 75%, 80%, 85%, 90%, 95%, 98%, or 100% identity to the nucleotide sequence of the open reading frame of Triticum aestivum aap13. According to some embodiments, the open reading frame of Triticum aestivum aap13 comprises the nucleotide sequence of SEQ ID NO: 8.

According to some embodiments, the AAP protein comprises an amino acid sequence having at least 75%, or at least 80%, or at least 85%, or at least 90%, or at least 95%, at least 98%, or 100% identity to the amino acid sequence of Triticum dicoccoides AAP3. According to certain embodiments, the Triticum dicoccoides AAP3 comprises the amino acid sequence set forth in SEQ ID NO: 9.

According to some embodiments, the polynucleotide encoding the AAP protein comprises a nucleotide sequence having at least 60%, 65%, 70%, 75%, 80%, 85%, 90%, 95%, 98%, or 100% identity to the nucleotide sequence of the open reading frame of Triticum dicoccoides aap3. According to some embodiments, the open reading frame of Triticum dicoccoides aap3 comprises the nucleotide sequence of SEQ ID NO: 10.

According to some embodiments, the AAP protein comprises an amino acid sequence having at least 75%, or at least 80%, or at least 85%, or at least 90%, or at least 95%, at least 98%, or 100% identity to the amino acid sequence of Pisum sativum AAP1. According to certain embodiments, the Pisum sativum AAP1 comprises the amino acid sequence set forth in SEQ ID NO: 11.

According to some embodiments, the polynucleotide encoding the AAP protein comprises a nucleotide sequence having at least 60%, 65%, 70%, 75%, 80%, 85%, 90%, 95%, 98%, or 100% identity to the nucleotide sequence of the open reading frame of Pisum sativum aap1. According to some embodiments, the open reading frame of Pisum sativum aap1 comprises the nucleotide sequence of SEQ ID NO: 12.

According to some embodiments, the expression and/or activity of the AAP and/or LHT is enhanced by at least 5%, 10%, 20%, 30%, 40%, 50% or more, compared with the expression and/or activity in the corresponding control plant grown under the same conditions. Each possibility represents a separate embodiment of the invention. According to certain exemplary embodiments, the number of AAP and/or LHT mRNA molecules or proteins is increased by at least 5%, 10%, 20%, 30%, 40%, 50% or more, compared with the number in a corresponding control plant grown under the same conditions. Each possibility represents a separate embodiment of the invention.

According to some embodiments, the plant is transformed with an exogenous polynucleotide encoding for AAP and/or LHT. According to some embodiments, the AAP and/or LHT1 is identical or similar to the endogenous AAP and/or LHT of the plant. According to other embodiments, the exogenous polynucleotide encoding for AAP and/or LHT is a heterologous polynucleotide derived from a plant of another species, genus or family.

According to some embodiments, the plant comprises two or more copies of the exogenous polynucleotide encoding for AAP and/or LHT.

According to some embodiments, the plant is transformed with an exogenous polynucleotide encoding for AAP and an exogenous polynucleotide encoding for LHT.

According to some embodiments, the AAP and/or LHT is overexpressed in the plant's leaves.

According to some embodiments, the polynucleotide encoding for AAP and/or LHT is operatively linked to a constitutive promoter. According to certain embodiments, the plant comprises a polynucleotide encoding for AAP and/or LHT, said polynucleotide is operably linked to Cauliflower mosaic virus 35S promoter. According to some embodiments, the promoter is the StUbi gene promoter or the AtPD1 promoter. According to additional embodiments, the promoter is a promoter of leaf senescence associated gene. According to additional embodiments, the promoter is a temporal promoter expressed during leaf senescence.

According to other embodiments, the polynucleotide encoding for AAP and/or LHT is operatively linked to an inducible promoter.

According to additional embodiments, the plant comprises a polynucleotide encoding for AAP and/or LHT, said polynucleotide is operably linked to a tissue-specific promoter. According to certain embodiments, the plant comprises a polynucleotide encoding for AAP and/or LHT1, said polynucleotide is operably linked to rbcS-3C promoter.

According to some embodiments, the plant comprises at least one polynucleotide encoding for AAP and/or at least one polynucleotide encoding for LHT.

According to other embodiments, the endogenous AAP and/or LHT is overexpressed. According to some embodiments, the promoter or enhancer of the endogenous AAP and/or LHT is replaced or modulated, such that the plant overexpresses said endogenous AAP and/or LHT.

According to some embodiments, the activity of AAP and/or LHT is increased. According to certain embodiments, the activity of AAP and LHT is increased.

According to some embodiments, the storage organ comprises an elevated amount of at least one free amino acid. According to certain embodiments, the amino acid is an essential amino acid. According to some embodiments, the storage organ comprises an elevated amount of at least one essential amino acid selected from the group consisting of lysine, methionine, histidine, isoleucine, leucine, phenylalanine, threonine, tryptophan, and valine. Each possibility represents a separate embodiment of the invention.

According to some embodiments, the amount of the at least one amino acid is increased by at least 5%, 10%, 20% or more, compared with a corresponding control plant.

According to some embodiments, the amino-acid composition in said plant is altered compared to the amino acid composition of a corresponding control plant. According to certain embodiments, said plant has a higher ratio of essential amino acids to other amino acids compared to the corresponding control plant.

According to some embodiments, the plant comprises an increased amount of total proteins in the storage organ. According to some embodiments, the total amount of the proteins in the storage organ is increased by at least 0.5% or 1%. According to some embodiments, the total amount of the proteins in the storage organ is increased by at least 2%. According to some embodiments, the total amount of the proteins in the storage organ is increased by at least 5%. According to some embodiments, the total amount of the proteins in the storage organ is increased by at least 10%, 15%, 20%, 30%, 40% or 50% compared with a corresponding control plant. Each possibility represents a separate embodiment of the invention.

According to some embodiments, the at least one protein present in an increased amount in the plant storage organ is an endogenous protein of said plant. According to certain exemplary embodiments, the plant is a potato and the protein is patatin.

According to some embodiments, the at least one protein present in an increased amount in the plant storage organ is a heterologous protein. According to some embodiments, the at least one protein is a heterologous protein other than AAP or LHT. According to some embodiments, the heterologous protein is a storage protein. According to some embodiments, the heterologous protein is a nutritional or pharmaceutical protein. According to these embodiments, the plant further comprises an exogenous polynucleotide encoding for the heterologous protein.

According to some embodiments, the heterologous protein is linked to a transit or signal peptide. According to some embodiments, the heterologous protein is linked to a plastid targeting peptide. According to certain embodiments, the transit peptide is a chloroplast transit peptide.

According to some embodiments, the exogenous polynucleotide encoding for the heterologous protein comprises a promoter selected from the group consisting of a constitutive promoter, an inducible promoter, a cell type-specific promoter, a developmental-stage specific promoter and a tissue-specific promoter.

According to some embodiments, the exogenous polynucleotide encoding for the heterologous protein comprises a promoter specifically active in the storage organ. According to certain embodiments, the exogenous polynucleotide encoding for the heterologous protein comprises a tuber-specific promoter. According to certain exemplary embodiments, the exogenous polynucleotide encoding for the heterologous protein comprises a patatin promoter.

According to some embodiments, the heterologous protein is an animal protein. According to some embodiments, the heterologous protein is a chicken egg protein. According to certain exemplary embodiments, the heterologous protein is ovalbumin.

According to some embodiments, the plant is Solanum tuberosum having an increased amount of at least one amino acid or protein in at least one of its tubers compared to a control plant. According to certain embodiments, the plant is Solanum tuberosum having an increased amount of total protein in at least one of its tubers compared to a control plant. According to additional embodiments, the plant is Solanum tuberosum having an increased amount of a heterologous protein in at least one of its tubers compared to a control plant.

A recombinant expression vector comprising a polynucleotide encoding for the heterologous protein described herein is also provided in some embodiments.

According to some embodiments, the transgenic plant tissue is hemizygous for the exogenous polynucleotide encoding for AAP and/or LHT. According to other embodiments, the transgenic plant tissue is homozygous for the exogenous polynucleotide encoding for AAP and/or LHT. According to certain embodiments, the plant is a tetraploid potato plant homozygous for the exogenous polynucleotide encoding for AAP and/or the exogenous polynucleotide encoding for LHT.

According to an additional aspect, the present invention provides a transgenic Solanum tuberosum plant comprising an increased amount of at least one essential amino acid and/or a protein in at least one of its tubers compared to a corresponding non-transgenic Solanum tuberosum plant grown under the same conditions, the plant having at least one cell having an enhanced expression and/or activity of at least one of AAP and LHT.

The AAP and LHT are as described herein. According to some embodiments, the AAP is AAP1, AAP3, TaAAP13, TdAAP3 or PsAAP1. According to some embodiments, the AAP is StAAP1 or AtAAP1. According to some embodiments, the LHT is LHT1 or LHT2. Each possibility represents a separate embodiment of the invention.

According to some embodiments, the transgenic Solanum tuberosum plant comprises an increased amount of a heterologous protein expressed in at least one of its tubers.

According to an additional aspect, the present invention provides a method of increasing the content of at least one amino acid or a protein in a storage organ of a plant, the method comprises enhancing the expression and/or activity of AAP and/or LHT in the plant relative to a corresponding control plant.

According to some embodiments, the AAP is selected from the group consisting of AAP1, AAP3, TaAAP13, TdAAP3, PsAAP1, OsAAP16 and AtAAP1.

According to some embodiments, the method comprises a step of enhancing the expression and/or activity of AAP1. According to some embodiments, the method comprises a step of enhancing the expression and/or activity of LHT. According to some embodiments, the method comprises a step of enhancing the expression and/or activity of AAP3. According to some embodiments, the method comprises a step of enhancing the expression and/or activity of TaAAP13. According to some embodiments, the method comprises a step of enhancing the expression and/or activity of TdAAP3. According to some embodiments, the method comprises a step of enhancing the expression and/or activity of PsAAP1. According to some embodiments, the method comprises a step of enhancing the expression and/or activity of one or more AAP1 and/or LHT and/or AAP3 and/or TaAAP13 and/or TdAAP3 and/or PsAAP1.

According to some embodiments, the method comprises a step of enhancing the expression and/or activity of at least one of AAP and LHT. According to some embodiments, the method comprises a step of enhancing the expression and/or activity of at least one of AAP1. AAP3, TaAAP13, TdAAP3, PsAAP1 StAAP1, AtAAP1, LHT1 and LHT2.

According to some embodiments, the method comprises a step of cultivating the plant under a defined condition.

According to some embodiments, the method comprises a step of growing the plant under conditions whereby enhancing the expression and/or activity increases the amount the amino acid and/or protein(s).

According to some embodiments, the method further comprises a step of transforming the plant with an additional exogenous polynucleotide encoding for a heterologous protein.

According to some embodiments, the method comprises a step of extracting or isolating the heterologous protein.

According to an additional aspect, the present invention provides a method of increasing the amount of at least one amino acid or protein in a storage organ of a plant, the method comprising:
(i) transforming the plant with at least one exogenous polynucleotide encoding for AAP and/or at least one exogenous polynucleotide encoding LHT; and
(ii) growing the plant.

According to some embodiments, the method comprises the step of (iii) transforming the plant with an exogenous polynucleotide encoding for an additional heterologous protein.

According to an additional aspect, the present invention provides a polynucleotide encoding for AAP and/or LHT, said polynucleotide comprises a promoter other than the natural promoter of the AAP or LHT gene.

According to some embodiments, a DNA construct comprising said polynucleotide is provided.

According to certain embodiment, the DNA construct comprises polynucleotides encoding for both AAP and LHT1.

According to some embodiments, the polynucleotide comprises a 35S promoter. According to some embodiments, the polynucleotide comprises a tissue specific promoter. According to some embodiments, the polynucleotide comprises a leaf specific promoter. According to additional embodiments, the polynucleotide comprises an inducible promoter.

According to additional embodiments, the polynucleotide comprises a promoter of leaf senescence associated gene or a temporal promoter expressed during leaf senescence leaf senescence promoter.

According to some embodiments, the promoter is of Solanum tuberosum, S. demissum, S. acaule, S. stoloniferum, S. phureja, S. gonicalyx, S. stenotomum, S. berthaultii, S. brevicaule, S. bukasovii, S. canasense, S. gourlayi, S. leptophyes, S. multidissectum, S. oplocense, S. sparsipilum, S. sucrense, S. venturii, or S. vernei. Each possibility represents a separate embodiment of the invention.

According to an additional aspect, the present invention provides a plant tuber comprising at least one exogenous polynucleotide encoding AAP and/or at least one exogenous polynucleotide encoding LHT or an active fragment thereof, wherein the plant tuber comprises an increased amount of at least one essential amino acid and/or total proteins compared to the amount in a corresponding plant tuber devoid of the at least one exogenous polynucleotide.

According to some embodiments, the plant tuber comprises an increased amount of total endogenous proteins.

According to some embodiments, the total amount of the proteins in the plant tuber is increased by at least 0.1%, 0.5%, or 1%. According to some embodiments, the total amount of the proteins in the plant tuber is increased by at least 5%, 10%, 15%, 20%, 30%, 40% or 50% compared with a corresponding control tuber.

According to some embodiments, the plant tuber has an increased amount of at least one essential amino acid by at least 0.1%, 0.5%, or 1% compared with a corresponding control tuber. According to some embodiments, the essential amino acids amount in the plant tuber is increased by at least 5%, 10%, 15%, 20%, 30%, 40% or 50% compared with a corresponding control tuber.

According to some embodiments, the plant tuber is a Solanum tuberosum tuber.

According to some embodiments, a food product comprising the plant tuber or part thereof is provided.

According to an additional aspect, the present invention provides a plant tuber comprising at least one exogenous polynucleotide that modulates the activity or expression of AAP and/or LHT, wherein the plant tuber comprises an increased amount of at least one essential amino acid and/or total proteins compared to the amount in a corresponding plant tuber devoid of the at least one exogenous polynucleotide.

Further embodiments and the full scope of applicability of the present invention will become apparent from the detailed description and drawings given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DRAWINGS

FIG. 1. Schematic diagram of a pCambia plasmid map, one of the plasmids used in example 1. The position of the AtAAP1 gene and StAAP1 promoter is shown.

FIG. 2. PCR amplification of StAAP1 sequence from 35S:StAAP1 expression plasmid extracted from potato plants after transformation.

### DETAILED DESCRIPTION OF THE INVENTION

The present invention relates to a genetic manipulation of the amino acids transport machinery in plants. In particular, the present invention relates to overexpression or recombinant expression of enzymes involved in the transport of amino acids from leaves to storage organs of plants, such as tubers. The methods of the invention in some embodiments increases the amount of the free amino acids, the plant's endogenous proteins, or recombinant proteins, in said storage organs.

Unexpectedly, it is now disclosed that the overexpression of AAP and/or LHT increases the amount of free amino acids and proteins in the plant storage organs. Without wishing to be bound by any particular theory or mechanism of action, the overexpression of the AAP and/or LHT increases the transportation of free amino acids to the phloem which are then accumulated in the storage organs. In some embodiments, the accumulated amino acids further enable the enhanced generation of endogenous or heterologous proteins within the storage organ.

According to one aspect, the present invention provides a transgenic plant having an edible storage organ, the plant comprising at least one cell having an enhanced expression and/or activity of at least one of amino acid permease (AAP) and Lysin histidine like transporter (LHT) and having: (1) an increased amount of at least one amino acid in the plant storage organ, and/or (2) an increased amount of at least one protein in said organ; compared to a corresponding non-transgenic plant.

According to an additional aspect, the present invention provides a method of increasing the content of at least one amino acid or protein in a storage organ of a plant, the method comprises enhancing the expression and/or activity of AAP and/or LHT in the plant relative to a control plant.

According to an additional aspect, the present invention provides a method of increasing the content of at least one heterologous protein in a storage organ of a plant, the method comprises enhancing the expression and/or activity of AAP and/or LHT in the plant relative to a control plant.

The heterologous protein is other than the AAP and LHT.

According to an additional aspect, the present invention provides a transgenic plant tuber comprising an increased amount of at least one essential amino acid and/or total proteins compared to a corresponding non-transgenic plant tuber under the same conditions, the tuber comprises an exogenous sequence of AAP and/or LHT or an active fragment thereof.

According to an additional aspect, the present invention provides a transgenic plant tuber comprising an increased amount of at least one essential amino acid compared to a corresponding non-transgenic plant tuber under the same conditions.

According to an additional aspect, the present invention provides a method of increasing the amount of at least one amino acid or protein in a storage organ of a plant, the method comprising:
(i) transforming the plant or part thereof with an exogenous polynucleotide encoding for AAP and/or an exogenous polynucleotide encoding for LHT; and
(ii) growing the plant.

According to certain embodiment, the plant is genetically modified to have enhanced expression and/or activity of the at least one amino acid permease (AAP) and/or the at least one Lysin histidine transporter (LHT).

According to some embodiments, the AAP is selected from the group consisting of AAP1, AAP3, TaAAP13, TdAAP3 and PsAAP1. According to some embodiments, the plant comprises at least one cell having an enhanced expression of AAP1. According to some embodiments, the plant comprises at least one cell having an enhanced expression of LHT. According to some embodiments, the plant comprises at least one cell having an enhanced expression of AAP3. According to some embodiments, the plant comprises at least one cell having an enhanced expression of TaAAP13. According to some embodiments, the plant comprises at least one cell having an enhanced expression of TdAAP3. According to some embodiments, the plant comprises at least one cell having an enhanced expression of PsAAP1. According to additional embodiments, the plant comprises at least one cell having an enhanced expression of both AAP and LHT.

AAP1, or Amino acid permease 1, is an amino acid-proton symporter, a stereospecific transporter with a broad specificity for histidine, glutamate and neutral amino acids. The aap1 gene encodes for AAP1 (e.g., GenBank: Y09825.2 (Solanum tuberosum)).

According to some embodiments, the AAP1 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 1.

The Solanum tuberosum AAP1 amino acid sequence is set forth in SEQ ID NO: 1. The open reading frame nucleotide sequence of aap1 is set forth in SEQ ID NO: 2.

According to some embodiments, the LHT is LHT1 or LHT2. LHT1, or Lysine histidine transporter 1, is an Amino acid-proton symporter. A transporter with a broad specificity for histidine, lysine, glutamic acid, alanine, serine, proline and glycine. Involved in both apoplastic transport of amino acids in leaves and their uptake by roots. The lht1 gene encodes for LHT1 (e.g., NCBI Reference Sequence: XM_006338609.2 (Solanum tuberosum)).

According to some embodiments, the LHT1 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 3.

The Solanum tuberosum LHT1 amino acid sequence is set forth in SEQ ID NO: 3. The open reading frame nucleotide sequence of lht1 is set forth in SEQ ID NO: 4.

AAP3, or Amino acid permease 3, is an amino acid-proton symporter, a stereospecific transporter with a broad specificity for histidine, glutamate and neutral amino acids. The aap3 gene encodes for AAP3 (e.g., GenBank: XM_006365475.2 (Solanum tuberosum)).

According to some embodiments, the AAP3 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 5.

The Solanum tuberosum AAP3 amino acid sequence is set forth in SEQ ID NO: 5. The open reading frame nucleotide sequence of aap3 is set forth in SEQ ID NO: 6.

TaAAP13, or Amino acid permease 13, is an amino acid-proton symporter, a stereospecific transporter with a broad specificity for histidine, glutamate and neutral amino acids. The Taaap13 gene encodes for TaAAP13 (e.g., GenBank: XM_44512810 (Triticum Aestivum)).

According to some embodiments, the TaAAP13 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 7.

The Triticum Aestivum AAP13 amino acid sequence is set forth in SEQ ID NO: 7. The open reading frame nucleotide sequence of aap3 is set forth in SEQ ID NO: 8.

TdAAP3, or Amino acid permease 3, is an amino acid-proton symporter, a stereospecific transporter with a broad specificity for histidine, glutamate and neutral amino acids. The Tdaap3 gene encodes for TdAAP3 (e.g., GenBank: XM_037573703.1 (Triticum dicoccoides)).

According to some embodiments, the TdAAP3 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 9.

The Triticum dicoccoides AAP3 amino acid sequence is set forth in SEQ ID NO: 9. The open reading frame nucleotide sequence of aap3 is set forth in SEQ ID NO: 10.

PsAAP1, or Amino acid trasporter 1, is an amino acid-proton symporter, a stereospecific transporter with a broad specificity for histidine, glutamate and neutral amino acids. The Psaap1 gene encodes for PsAAP1 (e.g., GenBank: AY956395 (Pisum sativum)).

According to some embodiments, the PsAAP1 comprises an amino acid sequence having at least 75%, 80%, 85%, 86%, 87%, 88%, 89%, 90%, 91%, 92%, 93%, 94%, 95%, 96%, 97%, 98%, 99%, or 100% identity to SEQ ID NO: 11.

The Pisum sativum AAP1 amino acid sequence is set forth in SEQ ID NO: 11. The open reading frame nucleotide sequence of Psaap1 is set forth in SEQ ID NO: 12.

According to some embodiments, the AAP is OsAAP16 (Oryza sativa; accession number-ABA95951.1).

According to some embodiments, the AAP is AtAAP1 (Arabidopsis thaliana; accession number—NP_176132.1, Gene ID: 842205).

It is to be understood that the invention also includes functional fragments of AAP or LHT1. A person skilled in the art would appreciate that it is possible to design a protein lacking one or several amino acids without deteriorating the function of the protein.

The term “expression” as used herein refers to the production of a protein product encoded by a gene.

The term “overexpression” as used herein refers to the production of a gene product in a transgenic plant that exceeds level of production in a non-modified or non-transgenic plant. The term “overexpressing” as used herein explicitly includes overexpression in part of the plant growth cycle or when the plant is grown under specific conditions. For example, if the polynucleotide encoding for AAP or LHT1 is under an inducible promoter, the plant is expected to overexpress the proteins when it has the right conditions or appropriate inducing factors.

The term “enhanced activity” as used herein refers to an elevated activity of the enzymes compared with the natural, endogenous enzyme. This enhanced activity may be achieved by overexpressing the endogenous enzyme, adding an additional, heterologous copy of the enzyme, or providing suitable conditions that increase its activity. The enhanced activity may be also achieved by incorporating a functional, active fragment of the enzyme.

The phrase “corresponding control plant” is as known in the art. For example, if the plant comprises enhanced expression and/or activity of AAP, the corresponding plant will have the same background without said enhanced expression and/or activity of AAP.

According to some embodiments, the plant comprises an exogenous nucleotide sequence encoding for AAP and/or LHT or a functional fragment thereof. The term “functional fragment thereof” as used herein refers to an amino acid sequence that is shorter in length than the full length of the protein sequence yet retains the activity of said protein. For example, in some embodiments, the functional fragment of the protein sequence comprises an amino acid sequence at least 100, at least 150, at least 200, at least 250, at least 300, at least 350, at least 400, at least 450, at least 500 or more amino acids in length and retains the activity of the protein.

The term “promoter” is used herein as known in the art. Promoter is a nucleic acid sequence located upstream or 5′ to a translational start codon of an open reading frame (ORF) of a gene and is involved in recognition and binding of RNA polymerase II and other proteins to initiate transcription. “Constitutive promoters” are functional in most or all tissues of a plant throughout plant development. Tissue-, organ- or cell-specific promoters are expressed only or predominantly in a particular tissue, organ, or cell type, respectively. For example, a “tuber promoter” is a native or non-native promoter that is functional in tuber cells.

As used herein, the terms “transgenic” and “transformed” are used interchangeably and refer to a plant, plant tissue or cell into which a foreign or recombinant DNA has been introduced. The term “foreign gene” or recombinant gene refers to any nucleic acid (e.g., gene sequence) that is introduced into the genome of an organism or tissue of an organism or a host cell by experimental manipulations, such as those described herein, and may include gene sequences found in that organism so long as the introduced gene does not reside in the same location, as does the naturally occurring gene.

As used herein, the term “coding” or “encoding” refers to the process by which a gene, through the mechanisms of transcription and translation, provides information to a cell from which a series of amino acids can be assembled into a specific amino acid sequence to produce an active enzyme. Because of the degeneracy of the genetic code, certain base changes in DNA sequence do not change the amino acid sequence of a protein.

Many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism may be tailored for improved expression in a different organism through codon optimization. According to some embodiments, AAP and/or LHT are codon-optimized for expression in the transgenic plant as described herein.

The terms “protein” and “polypeptide” are used interchangeably herein, and refer to a compound comprised of amino acid residues covalently linked by peptide bonds, or by means other than peptide bonds. A protein or polypeptide must contain at least 20 amino acids, and no limitation is placed on the maximum number of amino acids that can comprise a protein's or polypeptide's sequence. In one embodiment, a protein may comprise of more than one, e.g., two, three, four, five, or more, polypeptides, in which each polypeptide is associated to another by either covalent or non-covalent bonds/interactions. Polypeptides include any peptide comprising two or more amino acids joined to each other by peptide bonds or by means other than peptide bonds. “Polypeptides” and “proteins” include, for example, biologically active fragments, substantially homologous polypeptides, oligopeptides, homodimers, heterodimers, variants of polypeptides, modified polypeptides, derivatives, analogs, fusion proteins, among others.

The term “Endogenous”, as used herein, refers to any material from or naturally produced inside an organism, cell, tissue or system.

The term “Exogenous”, as used herein, refers to any material introduced to or produced outside of an organism, cell, tissue or system. Accordingly, “exogenous nucleic acid” refers to a nucleic acid that is introduced to or produced outside of an organism, cell, tissue or system. According to some embodiments, sequences of the exogenous nucleic acid are not naturally produced, or cannot be naturally found, inside the organism, cell, tissue, or system that the exogenous nucleic acid is introduced into.

The term “Heterologous”, as used herein, refers to any material from one species, when introduced to an organism, cell, tissue or system from a different species. According to some embodiments, a heterologous material also encompasses a material that includes portions from one or multiple species or portions that are non-naturally occurring. For example, in some embodiments, a nucleic acid encoding a fusion protein wherein a portion of the fusion protein is human, a portion of the fusion protein is plant, and a portion of the fusion protein is non-naturally occurring, and the nucleic acid is introduced to a plant cell, the nucleic acid is a heterologous nucleic acid. For example, the plant described herein is Solanum tuberosum and the heterologous protein is of another plant species or of an animal.

According to certain exemplary embodiments, the plant is selected from the group consisting of potato, batata, beet, soybean, peanut, maize, tomato, wheat, rice plant, Jicama, Cassava and sugar cane.

According to some embodiments, the plant is a tuberous plant. According to certain embodiments, the plant is selected from the group consisting of Solanum tuberosum, S. demissum, S. acaule, S. stoloniferum, S. phureja, S. gonicalyx, S. stenotomum, S. berthaultii, S. brevicaule, S. bukasovii, S. canasense, S. gourlayi, S. leptophyes, S. multidissectum, S. oplocense, S. sparsipilum, S. sucrense, S. venturii, and S. vernei. According to certain exemplary embodiments, the plant is Solanum tuberosum (potato).

According to some embodiments, the storage organ is selected from the group consisting of a tuber, root, seed, corm, rhizome, and fruit. Each possibility represents a separate embodiment of the invention. According to certain embodiments, the storage organ is a tuber.

The term “tuber” as used herein refers to a thickened, usually underground, food-storing organ that lacks both a basal plate and tunic-like covering, which corms and bulbs have. Roots and shoots grow from growth buds, called “eyes,” on the surface of the tuber. Potato tubers are produced by Solanum tuberosum, S. demissum, S. acaule, S. stoloniferum, S. phureja, S. gonicalyx, S. stenotomum, S. berthaultii, S. brevicaule, S. bukasovii, S. canasense, S. gourlayi, S. leptophyes, S. multidissectum, S. oplocense, S. sparsipilum, S. sucrense, S. venturii, and S. vernei.

The plants of the invention comprise an elevated amount of amino acids and/or proteins in their storage organs.

According to some embodiments, the plant storage organ, preferably tuber, comprises an elevated amount of at least one free amino acid. According to certain embodiments, the amino acid is an essential amino acid. According to some embodiments, the storage organ comprises an elevated amount of at least one essential amino acid selected from the group consisting of lysine, methionine, histidine, isoleucine, leucine, phenylalanine, threonine, tryptophan, and valine. Each possibility represents a separate embodiment of the invention.

According to some embodiments, the storage organ comprises an elevated amount of lysine. According to some embodiments, the storage organ comprises an elevated amount of methionine. According to some embodiments, the storage organ comprises an elevated amount of histidine. According to some embodiments, the storage organ comprises an elevated amount of isoleucine. According to some embodiments, the storage organ comprises an elevated amount of leucine. According to some embodiments, the storage organ comprises an elevated amount of phenylalanine. According to some embodiments, the storage organ comprises an elevated amount of threonine. According to some embodiments, the storage organ comprises an elevated amount of tryptophan. According to some embodiments, the storage organ comprises an elevated amount of valine.

The terms “elevated”, “increased”, or “enhanced” are used herein interchangeably and refer to an increased, for example, by at least 1%, 2%, 3%, 4%, 5%, 10% or more, for example at least 15%, 20%, 25%, 30%, 35%, 40% or 50% in comparison to a control or wild-type plant.

According to some embodiments, the amount of the at least one amino acid is increased by at least 2%, 5%, 10%, 15%, 20%, 25%, 30%, 35%, 40%, 45%, 50% or more, compared with a corresponding control plant. Each possibility represents a separate embodiment of the invention.

According to some embodiments, the amount of the total free amino acid is increased by at least 2%, 5%, 10%, 15%, 20%, 25%, 30%, 35%, 40%, 45%, 50% or more, compared with a corresponding control plant. Each possibility represents a separate embodiment of the invention.

According to some embodiments, the plant comprises an increased amount of at least one endogenous protein present in the storage organ. According to certain embodiments, the protein is patatin, a protease inhibitor, or starch synthesis phosphorylase.

The patatin gene encodes to a glycoprotein expressed in the potato tuber in large quantities and is related to the activity of lipid acyl hydrolase. A patatin gene promoter can regulate the potato tuber specific expression. The regulating factor located in bp −183 to −143 of the gene acts as a decisive factor for tuber specific expression induced by sugar (Liu et al., 1990, Mol. Genl Genet. 223:401-406). Further, a nucleus protein has been reported as a trans-acting factor that regulates the tuber specific expression of the patatin. According to some embodiments, the patatin gene promoter is utilized to the expression of the heterologous protein described herein.

According to some embodiments, the plant comprises an increased amount of total proteins in the storage organ, preferably in the tuber. According to some embodiments, the total amount of the proteins in the storage organ is increased by at least 1%. According to some embodiments, the total amount of the proteins in the storage organ is increased by at least 2%, 5%, 10%, 15%, 20%, 25%, 30%, 35%, 40%, 45% or 50%. Each possibility represents a separate embodiment of the invention.

It is to be understood that the increased amount of the amino acid(s) or protein(s) as described herein is compared to the amount of the amino acid(s) or protein(s) in a control storage organ having the same weight. Additionally or alternatively, an increased amount is compared per a weight or volume unit.

According to some embodiments, the amount is calculated as weight to weight (w/w). According to some embodiments, the amount is calculated as number of molecules to weight or volume. According to other embodiments the amount is calculated as percentage concentration.

According to certain embodiments, the storage organ comprises more than 7%, 7.5%, 8%. 8.5%, 9% or 10% of total amount/dry weight compared with a control storage organ.

According to certain embodiments, the storage organ comprises more than 20, 21, 22, 23, 24, 25, 26, 27, 28, 29, 30, 31, 32, 33, 34, 35, 36, 37, 38, 39, 40, or more mg/g of free amino acids compared with a control storage organ. Each possibility represents a separate embodiment of the invention.

The overexpression of AAP and/or LHT may be carried out by transforming the plant with an exogenous nucleic acid constructs encoding to the proteins. According to other embodiments, the endogenous AAP and/or LHT is overexpressed. According to some embodiments, the promoter or enhancer of the endogenous AAP and/or LHT is replaced or modulated, such that the plant overexpresses said endogenous AAP and/or LHT.

According to some embodiments, the methods of the present invention enable the increasing of the expression of a recombinant (e.g., heterologous) protein in the storage organ.

According to some embodiments, the at least one protein is a heterologous protein, said plant having an increased amount of the heterologous protein compared to a corresponding plant not overexpressing AAP and/or LHT. According to certain embodiments, the plant comprises an exogenous polynucleotide encoding for a heterologous protein.

According to some embodiments, the heterologous protein is linked to a transit or signal peptide. According to some embodiments, the heterologous protein is linked to a plastid targeting peptide. According to certain embodiments, the transit peptide is a chloroplast transit peptide. According to other embodiments, the protein is a secreted protein.

According to some embodiments, the exogenous polynucleotide encoding for the heterologous protein comprises a promoter selected from the group consisting of a constitutive promoter, an inducible promoter, a cell type-specific promoter, and a tissue-specific promoter.

According to embodiments, the exogenous polynucleotide encoding for the heterologous protein comprises a CaMV35S promoter. The term CaMV35S refers to cauliflower mosaic virus 35S promoter, a strong constitutive promoter.

According to certain embodiments, the plant comprises at least one cell having enhanced expression and/or activity of StAAP1, the StAAP1 is under the control of a non-native promoter. According to certain exemplary embodiments, the plant comprises at least one cell having enhanced expression and/or activity of StAAP1, the StAAP1 is under the control of a 35S promoter.

According to some embodiments, the heterologous protein is a secreted protein.

According to some embodiments, the heterologous protein is localized to the Golgi or endoplasmic reticulum (ER).

According to some embodiments, the heterologous protein is of an animal.

According to some embodiments, the heterologous protein is of an egg. According to some embodiments, the heterologous protein is selected from the group consisting of ovalbumin, ovotransferrin, ovomucoid, ovomucin, and lysozyme.

According to other embodiments, the heterologous protein is selected from a group consisting of interleukin-2, hirudin, insulin, interferons, lactoferrin, hemoglobin, erythropoietin, epidermal growth factor, anthrax vaccines, cholera vaccine, DPT vaccine, hib vaccine, hepatitis A vaccine, hepatitis B vaccine, hepatitis C vaccine, HPV vaccine, influenza vaccine, Japanese Encephalitis vaccine, MMR vaccine, MMRV vaccine, pneumococcal conjugate vaccine, pneumococcal polysaccharide vaccine, polio vaccine, rotavirus vaccine, smallpox vaccine, tuberculosis vaccine, typhoid vaccine, yellow fever vaccine, parvovirus vaccine, distemper vaccine, adenovirus vaccine, parainfluenza vaccine, bordetella vaccine, rabies vaccine, leptospirosis vaccine, lyme vaccine, corona vaccine, round/hookworm vaccine, dewormer vaccine, RNFN vaccine, and HIV vaccine. Each possibility represents a separate embodiment of the invention.

All technical terms used herein are terms commonly used in biochemistry, molecular biology and agriculture, and can be understood by one of ordinary skill in the art to which this invention belongs. Those technical terms can be found e.g. in: Molecular Cloning: A Laboratory Manual, 4th ed., Green and Sambrook, Cold Spring Harbor Laboratory Press, Cold Spring Harbor, N. Y., 2012; Current Protocols in Molecular Biology, ed. Ausubel et al., Greene Publishing Associates and Wiley-Interscience, New York, 1988 (with periodic updates); Short Protocols In Molecular Biology: A Compendium of Methods From Current Protocols in Molecular Biology, 5^{th} ed., vol. 1-2, ed. Ausubel et al., John Wiley & Sons, Inc., 2002; Genome Analysis: A Laboratory Manual, vol. 1-2, ed. Green et al., Cold Spring Harbor Laboratory Press, Cold Spring Harbor, N.Y., 1997. Methodology involving plant biology techniques is described herein and is described in detail in books such as Biochemistry and Molecular Biology Of Plants, Buchanan, et al., John Wiley Sons Inc, 2005.

In order to overexpress AAP and/or LHT, and the expression of a heterologous protein in the plant, a nucleic acid sequence (a polynucleotide) encoding said proteins may be transformed to the plant by any method known in the art.

A nucleic acid construct can be introduced into any plant cell using a suitable genetic engineering technique. Both monocotyledonous and dicotyledonous angiosperm or gymnosperm plant cells may be genetically engineered in various ways known to the art. Exemplary methodology includes but is not limited to transformation, electroporation, particle gun bombardment, calcium phosphate precipitation, and polyethylene glycol fusion, transfer into germinating pollen grains, direct transformation (Lorz et al., Mol. Genet. 199:179-182 (1985)), and other methods known to the art.

Numerous methods for plant transformation have been developed including biological and physical plant transformation protocols. See, for example, Miki, et al., “Procedures for Introducing Foreign DNA into Plants,” in Methods in Plant Molecular Biology and Biotechnology, Glick and Thompson Eds., CRC Press, Inc., Boca Raton, pp. 67-88 (1993). In addition, expression vectors and in-vitro culture methods for plant cell or tissue transformation and regeneration of plants are available. See, for example, Gruber, et al., “Vectors for Plant Transformation,” in Methods in Plant Molecular Biology and Biotechnology, Glick and Thompson Eds., CRC Press, Inc., Boca Raton, pp. 89-119 (1993).

Agrobacterium-mediated Transformation-One method for introducing an expression vector into plants is based on the natural transformation system of Agrobacterium. See, for example, Horsch, et al., Science, 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci., 10:1 (1991). Descriptions of Agrobacterium vector systems and methods for Agrobacterium-mediated gene transfer are well known in the art.

According to some embodiments, an Agrobacterium species such as A. tumefaciens and A. rhizogenes are used. Briefly, Agrobacterium may be transformed with a plant expression vector via, e.g., electroporation, after which the Agrobacterium is introduced to plant cells via, e.g., the leaf-disk method.

The Agrobacterium transformation methods discussed above are known to be useful for transforming dicots. Several studies have shown the use of vacuum infiltration for Agrobacterium-mediated transformation.

Numerous processes are available to introduce DNA into a plant host cell. In many processes it is necessary that the nucleotide sequences to be introduced occur in cloning and/or expression vectors. Vectors are essentially plasmids, cosmids, viruses, bacteriophages, shuttle vectors, and other vectors commonly used in genetic engineering. Vectors can have other functional units which stabilize the vector in a host organism and/or make its replication possible. Vectors can also contain regulatory elements functionally linked to the nucleotide sequence obtained and which allow expression of the nucleotide sequence in a host organism. Such regulatory units can be promoters, enhancers, operators and/or transcription termination signals. Vectors also frequently contain marker genes which allow selection of the host organisms containing them, such as antibiotic resistance genes.

Processes for introducing DNA into plant cells include transformation of plant cells with Agrobacterium as transforming agents, protoplast fusion, microinjection, electroporation of DNA, introduction of DNA by means of biolistic methods and other possibilities. The processes of microinjection and electroporation of DNA into plant cells do not themselves place any special requirements on the plasmids to be used. Simple plasmids can be used, such as pUC derivatives. However, if whole plants are to be regenerated from cells transformed in that manner, a selectable marker should be present.

Depending on the process used to introduce coding nucleotide sequences into the plant cells, it may be necessary for the vector to contain other DNA sequences. For example, if the Ti or R1 plasmid is used to transform plant cells, it is necessary for at least the right border sequence, and often both the right and left border sequences of the Ti and R1 plasmid cells to be linked as flank regions with the genes being introduced. When Agrobacterium is used for transformation, the DNA being introduced must be cloned in special plasmids, in either an intermediary vector or a binary vector. Because of sequences homologous with sequences in the T-DNA, intermediary vectors can be integrated into the Ti or R1 plasmid of Agrobacterium through homologous recombination. Those also contain the vir region required for transfer of the T-DNA. Intermediary vectors cannot replicate in agrobacteria. The intermediary vector can be transferred to Agrobacterium tumefaciens by means of a helper plasmid. In contrast, binary vectors can replicate both in agrobacteria and in E. coli. They contain a gene for a selection marker and a linker or polylinker framed by the right and left T-DNA border regions. Binary vectors can be transformed directly into agrobacteria. The Agrobacterium which serves as the host cell should contain a plasmid carrying a vir region. This vir region is necessary for the transfer of the T-DNA into the plant cell. The Agrobacterium transformed in that way is used to transform plant cells. Use of T-DNA to transform plant cells is described in the following publications, among others: EP-A-120 516; Hoekema: The Binary Plant Vector System, Offsetdrukkerej Kanters. B. V., Alblasserdam (1985), Chapter V; Fralej et al., Crit. Rev. Plant. Sci., 4, 1-46, and An et al., EMBO J., 4 (1985), 277-287). Plant explants can be co-cultivated with the agrobacterium to transfer the DNA into the plant cells. The whole plants can be regenerated from the infected plant material, such as leaf fragments, stem segments, roots, protoplasts, or plant cells cultivated in suspension, in a suitable medium which contains antibiotics or biocides to select transformed cells.

Microprojectile Bombardment—An additional method for transforming DNA segments to plant cells is microprojectile bombardment. In this method, microparticles may be coated with DNA and delivered into cells by a propelling force. Exemplary particles include those comprised of tungsten, gold, platinum, and the like.

Methods such as microprojectile bombardment or electroporation are carried out with “naked” DNA where the expression cassette may be simply carried on any E. coli-derived plasmid cloning vector. In the case of viral vectors, it is desirable that the system retain replication functions, but lack functions for disease induction.

For bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Using techniques set forth herein, one may obtain up to 1000 or more foci of cells transiently expressing a marker gene. The number of cells in a focus which express the exogenous gene product 48 hours post-bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. Execution of such routine adjustments will be known to those of skill in the art.

Electroporation—where one wishes to introduce DNA by means of electroporation, it is contemplated that the method of Krzyzek et al. (U.S. Pat. No. 5,384,253) may be advantageous. In this method, certain cell wall-degrading enzymes, such as pectin-degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin-degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

It is contemplated that in some instances DNA precipitation onto metal particles would not be necessary for DNA delivery to a recipient cell using microprojectile bombardment. It is further contemplated that particles may contain DNA rather than be coated with DNA. Hence it is proposed that particles may increase the level of DNA delivery but are not, in and of themselves, necessary to introduce DNA into plant cells.

Suitable methods for transformation of host plant cells for use with the current invention include virtually any method by which DNA can be introduced into a cell (for example, where a recombinant DNA construct is stably integrated into a plant chromosome) as described hereinabove and are well known in the art. Another exemplary method for introducing a recombinant DNA construct into plants is insertion of a recombinant DNA construct into a plant genome at a pre-determined site by methods of site-directed integration. Site-directed integration may be accomplished by any method known in the art, for example, by use of zinc-finger nucleases, engineered or native meganucleases, TALE-endonucleases, or an RNA-guided endonuclease (for example a CRISPR/Cas9 system). Transgenic plants can be regenerated from a transformed plant cell by the methods of plant cell culture or by taking a cutting from a transgenic plant and rooting the cutting to establish a vegetative clone of the transgenic plant. A transgenic plant homozygous with respect to a transgene (that is, two allelic copies of the transgene) can be obtained by self-pollinating (selfing) a transgenic plant that contains a single transgene allele with itself, for example an R0 plant, to produce R1 seed. One fourth of the R1 seed produced will be homozygous with respect to the transgene. Plants grown from germinating R1 seed can be tested for zygosity, typically using a SNP assay, DNA sequencing, or a thermal amplification assay that allows for the distinction between heterozygotes and homozygotes, referred to as a zygosity assay.

The genetic constructs used in the present invention may generally contain a suitable promoter which functions in plant cells, a suitable terminator such as nopaline synthetic enzyme gene terminator, other elements useful for regulating the expression and marker genes suitable for selecting the transformant such as drug-resistant genes, e.g. genes resistant to kanamycin, G418 or hygromycin in addition to the intended gene. The promoter contained in the genetic construct may be a constitutive promoter, an organ-specific promoter or a developmental stage-specific promoter and can be suitably selected depending on the host, gene, desired expression level, organ for the expression, developmental stage, etc.

Most endogenous genes have regions of DNA that are known as promoters, which regulate gene expression. Promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide base pairs. Promoter sequences also contain regulatory sequences such as enhancer sequences that can influence the level of gene expression. Some isolated promoter sequences can provide for gene expression of the AAP, LHT1 or heterologous protein as described herein, that is a DNA encoding to a protein different from the native or homologous DNA.

Promoter sequences are also known to be strong or weak, or inducible. A strong promoter provides for a high level of gene expression, whereas a weak promoter provides for a very low level of gene expression. An inducible promoter is a promoter that allows gene expression to be turned on and off in response to an exogenously added agent, or to an environmental or developmental stimulus. Promoters can also provide for tissue specific or developmental regulation. An isolated promoter sequence that is a strong promoter for heterologous DNAs is advantageous because it provides for a sufficient level of gene expression for easy detection and selection of transformed cells and provides for a high level of gene expression when desired.

Expression cassettes generally include, but are not limited to, a plant promoter such as the CaMV 35S promoter, or others such as CaMV 19S, nos, Adh1, sucrose synthase, α-tubulin, ubiquitin, actin, cab, PEPCase or those associated with the R gene complex. Further suitable promoters include the poplar xylem-specific secondary cell wall specific cellulose synthase 8 promoter, cauliflower mosaic virus promoter, the Z10 promoter from a gene encoding a 10 kDa zein protein, a Z27 promoter from a gene encoding a 27 kDa zein protein, inducible promoters, such as the light inducible promoter derived from the pea rbcS gene and the actin promoter from rice. Seed specific promoters, such as the phascolin promoter from beans, may also be used. Further suitable promoters include Tuber specific promoters of lacase gene (https://www.ncbi.nlm.nih.gov/nuccore/JX983655.1) and B33 (patatin promoter) (https://www.ncbi.nlm.nih.gov/nuccore/X14483.1). Also, leaf senescence promoter S1SuT1 (Solanum Lycopersicon sucrose transporter) (https://www.ncbi.nlm.nih.gov/nuccore/AF176638.1) and the 1.7 KB upstream to ATG region of StAAP1.

Alternatively, novel tissue specific promoter sequences may be employed in the practice of the present invention. cDNA clones from a particular tissue can be isolated and those clones which are expressed specifically in that tissue are identified, for example, using Northern blotting. The promoter and control elements of corresponding genomic clones can then be localized using techniques well known to those of skill in the art.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. The transformation is carried out under conditions directed to the plant tissue of choice.

According to the present invention, an intended plant can be obtained by introducing and manipulating a genetic construct as disclosed herein into various plant cells including, but are not limited to, protoplasts, tissue-cultured cells, tissues and organ explants, pollens, embryos and whole plant bodies. From the plants manipulated according to the embodiment of the present invention, the intended transgenic plant is selected or screened by an approach and method as known in the art. An individual plant body may be regenerated from the isolated transformant. Methods of regenerating individual plant bodies from plant cells, tissues or organs for various species are well known by those skilled in the art.

The transformed plant cells, calli, tissues or plants may be identified and isolated by selecting or screening the characters encoded by marker genes contained in the genetic construct used for the transformation. For example, the selection may be conducted by growing a manipulated plant in a medium containing a repressive amount of antibiotic or herbicide, to which the introduced genetic construct can impart the resistance. Further, the transformed plant cells and plants may be identified by the screening with reference to the activity of visible marker genes (such as β-glucuronidase genes, luciferase genes, B genes or Cl genes) which may be present in the transgenic nucleic acid construct of the present invention. The methods of the selection and screening are well known by those skilled in the art.

It is further contemplated that combinations of screenable and selectable markers may be useful for identification of transformed cells. For example, selection with a growth inhibiting compound, such as bialaphos or glyphosate at concentrations below those providing 100% inhibition followed by screening of growing tissue for expression of a screenable marker gene such as luciferase would allow one to recover transformants from cell or tissue types that are not amenable to selection alone. Selectable marker genes include, but are not limited to, Kan, GFP, EGFP, GUS, LUX, CAH, SPT, NPTII, HPT, APHIV, BAR, PAT, CHS, AHAS and flavonoid synthesis genes.

An exemplary embodiment of methods for identifying transformed cells involves exposing the cultures to a selective agent, such as a metabolic inhibitor, an antibiotic, herbicide or the like. Cells which have been transformed and have stably integrated a marker gene conferring resistance to the selective agent used, will grow and divide in culture. Sensitive cells will not be amenable to further culturing.

Regeneration and seed production: cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA⁺², 4-D or possibly picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber as known in the art. Plants can be matured either in a growth chamber or greenhouse. In some embodiments, plants are regenerated from about 6 weeks to 10 months after a transformant is identified, depending on the initial tissue. During regeneration, cells are grown on solid media in tissue culture vessels. After the regenerating plants have reached the stage of shoot and root development, they may be transferred to a greenhouse for further growth and testing. Mature plants are then obtained from cell lines that are known to express the trait.

A variety of promoters specifically active in the storage organs, such as roots and tubers, can be used to express the heterologous protein of the present invention. Examples of tuber-specific promoters include, but are not limited to, the class I and II patatin promoters. the promoter for the potato tuber ADPGPP genes, the sucrose synthase promoter, and the promoter for the major tuber proteins including the 22 kd protein complexes and proteinase inhibitors.

A variety of promoters can be used to overexpress AAP or LHT. Examples of leaf-specific promoters include, but are not limited to the ribulose biphosphate carboxylase (RBCS or RuBISCO) promoters; the light harvesting chlorophyll a/b binding protein gene promoter; and the Arabidopsis thaliana myb-related gene promoter (Atmyb5).

According to some embodiments, the promoter is a leaf senescence promoter. According to certain exemplary embodiments, the promoter is S1SUT1, StAAP1, or AtAAP1 promoter.

According to some embodiments, the promoter is StLHT1 promoter.

According to other embodiments, the promoter is a tuber promoter. According to certain embodiments, the promoter is a GBSS or patatin promoter.

The transgenic plants, progeny, seeds, plant cells, and plant parts of the invention may also contain one or more additional traits. Additional traits may be introduced by crossing a plant containing a transgene comprising the recombinant DNA molecules provided by the invention with another plant containing one or more additional trait(s). As used herein, “crossing” means breeding two individual plants to produce a progeny plant. Two plants may thus be crossed to produce progeny that contain the desirable traits from each parent. As used herein “progeny” means the offspring of any generation of a parent plant, and transgenic progeny comprise a DNA construct provided by the invention and inherited from at least one parent plant. Additional trait(s) also may be introduced by co-transforming a DNA construct for that additional transgenic trait(s) with a DNA construct comprising the recombinant DNA molecules provided by the invention (for example, with all the DNA constructs present as part of the same vector used for plant transformation) or by inserting the additional trait(s) into a transgenic plant comprising a DNA construct provided by the invention or vice versa (for example, by using any of the methods of plant transformation or genome editing on a transgenic plant or plant cell). Such additional traits include, but are not limited to, increased insect resistance, increased water use efficiency, increased yield performance, increased drought resistance, increased seed quality, improved nutritional quality, hybrid seed production, and herbicide-tolerance, in which the trait is measured with respect to a wild-type plant. Exemplary additional herbicide-tolerance traits may include transgenic or non-transgenic tolerance to one or more herbicides such as ACCase inhibitors (for example aryloxyphenoxy propionates and cyclohexanediones), ALS inhibitors (for example sulfonylureas, imidazolinones, triazolopyrimidines, and triazolinones) EPSPS inhibitors (for example glyphosate), synthetic auxins (for example phenoxys, benzoic acids, carboxylic acids, semicarbazones), photosynthesis inhibitors (for example triazines, triazinones, nitriles, benzothiadiazoles, and ureas), glutamine synthesis inhibitors (for example glufosinate), HPPD inhibitors (for example isoxazoles, pyrazolones, and triketones), PPO inhibitors (for example diphenylethers, N-phenylphthalimide, aryl triazinones, and pyrimidinediones), and long-chain fatty acid inhibitors (for example chloroacetamindes, oxyacetamides, and pyrazoles), among others. Exemplary insect resistance traits may include resistance to one or more insect members within one or more of the orders of Lepidoptera, Coleoptera, Hemiptera, Thysanoptera, Diptera, Hymenoptera, and Orthoptera, among others. Such additional traits are well known to one of skill in the art; for example, and a list of such transgenic traits is provided by the United States Department of Agriculture's (USDA) Animal and Plant Health Inspection Service (APHIS).

The nucleic acid constructs usable in the present invention can be prepared by the methods well known by those skilled in the art. For example, the recombinant DNA techniques which can be used for isolating the components of a construct, determining their features, handling them, and generating the construct, can be found in, for example, Sambrook et al., Molecular cloning-Laboratory manual, the second edition (Cold Spring Harbor Laboratory Press). When a nucleotide sequence of a desired component is known, it is advantageous not to isolate it from a biological source but to synthesize it. In some cases, the desired component may be advantageously produced by polymerase chain reaction (PCR) amplification.

Physical methods and biochemical methods can be employed for identifying plants containing the genetic construct of the present invention or plant cells transformed with the construct. Examples of the methods include: (1) Southern analysis or PCR amplification for detecting and determining the structure of recombinant DNA insert; (2) Northern blotting, S1 RNase protection, primer elongation PCR amplification or reverse transcriptase PCR (RT-PCR) amplification for detecting and determining the RNA transcription product of genetic construct; and (3) when the genetic construct is a protein, protein gel electrophoresis, western blotting, immune precipitation, enzyme immunoassay, etc. but the methods are not limited to them. These assay methods are well known by those skilled in the art.

As used herein, the term “about” when combined with a value refers to +10% of the reference value.

As used herein the singular forms “a”, “an”, and “the” include plural references unless the context clearly dictates otherwise. Thus, for example, reference to “a compound” includes a plurality of such compounds. It should be noted that the term “and” or the term “or” are generally employed in their sense including “and/or” unless the context clearly dictates otherwise.

As used herein, the term “comprising” means “including but not limited to”.

As used herein, the term “and/or” refers to and encompasses any and all possible combinations of one or more of the associated listed items, as well as the lack of combinations when interpreted in the alternative (“or”).

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Methods

### Agrobacterium Mediated Transformation

1. Cut young dark green leaves were from 3-4-weeks-old sterile grown Solanum tuberosum Desirée′ plants, and placed in a sterile petri dish or on sterile paper.
2. Remove petiole and cut leaves.
3. Cut the leaves edges from all sides and cut the leaves to squares of approximately 1×1 cm (depends on leaf size) to enable agrobacterium infection through the wounding.
4. Transfer to a new petri dish containing liquid MS3s media (4.4 g/L MS; 30 g/L Sucrose; pH=5.8).
5. Grow agrobacterium containing the desired expression plasmid over-night in 3 ml LB with Rif (25) and the expression plasmid selection antibiotic (e.g., KAN 50, Spec 50).
6. Dilute over-night culture (1:10) to fresh LB medium with appropriate antibiotics and acetosyringone (100 μM) and grow 4-6 hours 28° C. 200 rpm (O.D. 0.5-1.6).
7. Centrifuge 10 minutes, 4000 rpm at room temp and resuspend in MS3s with acetosyringone (100 μM) to a final OD of 0.5-0.8.
8. Transfer cut leaves to the agrobacterium culture and gently shake in dark for 30 min at room temperature.
9. Dry the leaves for 5 min on a sterile Whatman paper and place faced-down onto co-cultivation media plates (DEI− MS3s+ acetosyringone 200 μM).
10. Place the plates in dark for 48 h in the growth chamber.
11. After 48 h of co-cultivation, place infected leaves faced-down on DEII plates (4.4 g/L MS; 30 g/L Sucrose; 5 mg/L NAA; 2 mg/L BAP+selection antibiotics+claforan 500 mg/L; pH=5.8) for 14-21 days in the growth chamber.
12. After Callus appears, place leaves onto DEIII plates (4.4 g/L MS; 30 g/L Sucrose; 2 mg/L Zeatin riboside; 0.02 mg/L NAA; 0.02 mg/L GA3+antibiotics; pH=5.8). Transfer to fresh media every 2-3 weeks, until shoots formed (about 6 weeks).
13. When shoots reach 1-2 cm in length, cut them from the callus and transfer to container containing “roots induction media” (MS3s+500 mg/L claforan+selection antibiotic) for plantlet formation and transgenic plant growth.
14. Transfer the plant to small pot inside a closed and transparent container for hardening for 1-2 weeks.
15. Open the container and put the cover in a way that will cover most of the box. Open it a little bit more every day, for 3 days until removing the cover completely.
16. Transfer the plant to a greenhouse.

### Example 1: Stable Transformation of AAP1 to Solanum tuberosum

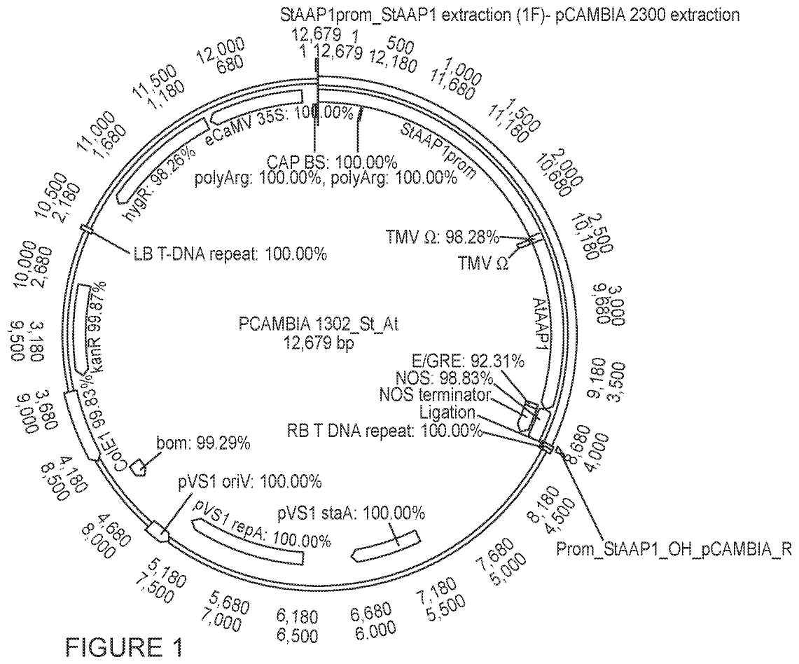

AAP1 coding sequence was inserted into different pCambia plasmids (e.g. 1302 and 2300 pCambia of abcam). The plasmids were further inserted with specific promoter, translation enhancer and a terminator sequence. One of the plasmids that were used, pCambia with AtAAP1 under the promoter of StAAP1, is shown in FIG. 1. The plasmids were transformed into agrobacterium using a standard transformation protocol as known in the art.

Young dark green leaves were cut from a 3-4 week old sterile grown Solanum tuberosum Desirée' plants, and placed in a sterile petri dish. Leaves were cut through the middle-rip one- or two-fold depending on the leaf size, to enable agrobacterium infection through the wounding. Petiole were removed and cut leaves were transferred to a new petri dish containing MS1 media (4.4 gr/L MS; 30 gr/L Sucrose; pH=5.8).

Agrobacterium culture containing the plasmid was grown over-night and resuspended to OD₆₀₀=0.4 in a fresh MS2 media (MS1; 100 μM Acetosyringone). Cut leaves were transferred to the agrobacterium culture and were gently shake in dark for 20 min at room temperature. Leaves were dried for 10 min on a sterile Whatman paper and placed faced down onto co-cultivation media plates (MS1; 200 μM Acetosyringone; pH=5.8). Plates were placed in dark for 48 h at 21-23° C.

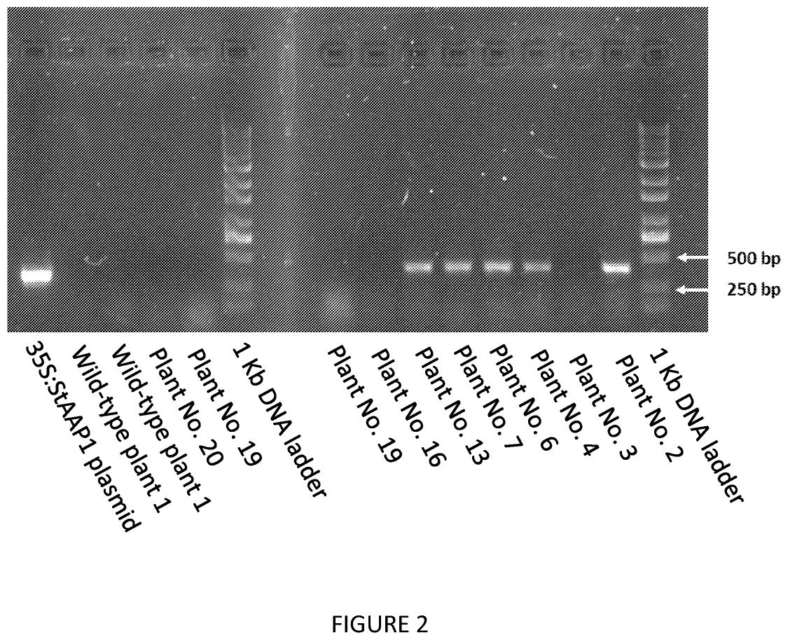

After 48 h of co-cultivation, infected leaves were placed faced-down on callus induction media plates (MS1; 5 mg/L NAA; 0.1 mg/L BAP; appropriate antibiotics) for 8-10 days at 21-23° C. After Callus appeared (˜10 days), leaves were placed onto shoot induction media plates (MS1; 2 mg/L Zeatin-ribiside; 0.02 mg/L NAA; 0.2 mg/L GA3; appropriate antibiotics), and transferred to fresh media every 8 days, until shoots formed (about 6 weeks). When shoots reached 1-2 cm length, they were cut from the callus and transferred to a jar containing roots induction media (MS1+appropriate antibiotics) for plantlet formation and transgenic plant growth. Transformation was confirmed using PCR (FIG. 2).

### Example 2: Increased Amount of Amino Acids and Total Protein

The transgenic plants are grown and harvest. Total amount of proteins, amino acids, and the composition of the amino acids in the tuber are measured as known in the art. The transgenic plants are used for expressing heterologous proteins and their amount is measured.

### Sequences:

SEQ ID NO: 1—Solanum tuberosum AAP1—Amino acids
SEQ ID NO: 2—Solanum tuberosum aap1—Nucleic acids
SEQ ID NO: 3—Solanum tuberosum LHT1—Amino acids
SEQ ID NO: 4—Solanum tuberosum lht1—Amino acids
SEQ ID NO: 5—Solanum tuberosum AAP3—Amino acids
SEQ ID NO: 6—Solanum tuberosum aap3—Nucleic acids
SEQ ID NO: 7—Triticum aestivum TaAAP13—Amino acids
SEQ ID NO: 8—Triticum aestivum Taaap13—Nucleic acids
SEQ ID NO: 9—Triticum dicoccoides TdAAP3—Amino acids
SEQ ID NO: 10—Triticum dicoccoides Tdaap3—Nucleic acids
SEQ ID NO: 11—Pisum sativum PsAAP1—Amino acids
SEQ ID NO: 12—Pisum sativum Psaap1—Nucleic acids

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A tuberous plant comprising at least one cell having an enhanced expression and/or activity of at least one amino acid permease (AAP) and/or at least one Lysin histidine like transporter (LHT), compared to the expression and/or activity in a corresponding control plant, and having: (1) an increased amount of at least one free amino acid in the plant tuber, and/or (2) an increased amount of at least one protein in said tuber; compared to the amount in the corresponding control plant.

2. The plant of claim 1, wherein the at least one cell has an enhanced expression and/or activity of AAP.

3. The plant of claim 1, wherein the AAP protein comprises an amino acid sequence having at least 75%, or at least 80%, or at least 85%, or at least 90%, or at least 95%, at least 99%, or 100% identity to the amino acid sequence of Solanum tuberosum AAP1 set forth in SEQ ID NO: 1.

4. The plant of claim 1, wherein AAP is selected from the group consisting of amino acid permease 1 (AAP1), amino acid permease 3 (AAP3), Triticum aestivum amino acid permease 13 (TaAAP13), Triticum dicoccoides amino acid permease 3 (TdAAP3), and Pisum sativum amino acid transporter (PsAAP1).

5. The plant of claim 1, wherein the at least one cell has an enhanced expression and/or activity of LHT.

6. The plant of claim 1, wherein the LHT is LHT1 or LHT2.

7. The plant of claim 1, wherein the LHT protein comprises an amino acid sequence having at least 75%, or at least 80%, or at least 85%, or at least 90%, or at least 95%, or at least 99%, or 100% identity to the amino acid sequence of Solanum tuberosum LHT1 set forth in SEQ ID NO: 3.

8. The plant of claim 1, wherein the at least one cell has an enhanced expression and/or activity of both AAP and LHT.

9-11. (canceled)

12. The plant of claim 1, wherein the plant is selected from the group consisting of Solanum tuberosum, S. demissum, S. acaule, S. stoloniferum, S. phureja, S. gonicalyx, S. stenotomum, S. berthaultii, S. brevicaule, S. bukasovii, S. canasense, S. gourlayi, S. leptophyes, S. multidissectum, S. oplocense, S. sparsipilum, S. sucrense, S. venturii, and S. vernei.

13. The plant of claim 1, wherein the at least one free amino acid is an essential amino acid.

14. (canceled)

15. The plant of claim 1, wherein the amount of the at least one free amino acid is increased by at least 1%, 5%, 10%, 20% or more, compared with the corresponding control plant.

16. The plant of claim 1, wherein the plant comprises an increased amount of total proteins in the tuber.

17. The plant of claim 1, wherein the at least one protein present in increased amount in the plant tuber is a heterologous protein.

18. The plant of claim 1, wherein said plant comprises an exogenous polynucleotide encoding for the heterologous protein, said exogenous polynucleotide comprises a promoter selected from the group consisting of a constitutive promoter, an inducible promoter, a cell type-specific promoter, and a tissue-specific promoter.

19. The plant of claim 18, wherein the exogenous polynucleotide encoding for the heterologous protein comprises a tuber-specific promoter.

20. The plant of claim 17, wherein the heterologous protein is a nutritional or pharmaceutical protein.

21. The plant of claim 17, wherein the heterologous protein is an animal protein.

22-24. (canceled)

25. A method of increasing the content of at least one amino acid and/or a protein in a plant tuber, the method comprising enhancing the expression and/or activity of AAP and/or LHT in the plant relative to a control plant.

26-28. (canceled)

29. A heterologous protein produced by a method according to claim 25.

30. A plant tuber comprising at least one exogenous polynucleotide encoding AAP and/or at least one exogenous polynucleotide encoding LHT or an active fragment thereof, wherein the plant tuber comprises an increased amount of at least one essential amino acid and/or total proteins compared to the amount in a corresponding plant tuber devoid of the at least one exogenous polynucleotide.

31-33. (canceled)
